(19) United States
(12) Patent Application Publication
(10) Pub. No.: US 2016/0339183 A1
(43) Pub. Date: Nov. 24, 2016

(54) INJECTOR FOR BONE REGENERATION

(71) Applicant: National Taiwan University, Taipei (TW)

(72) Inventors: Hsiao-Cheng TSAI, Taipei (TW); Yi-Chen LI, Taipei (TW); Tai-Horng YOUNG, Taipei (TW); Min-Huey CHEN, Taipei (TW)

(73) Assignee: National Taiwan University, Taipei (TW)

(21) Appl. No.: 14/873,584

(22) Filed: Oct. 2, 2015

(30) Foreign Application Priority Data
May 20, 2015 (TW) 104116007

(51) Int. Cl. A61M 5/32; A61M 5/315; A61C 19/06; A61M 5/34

(52) U.S. Cl. CPC A61M 5/329; A61M 5/343; A61M 5/349; A61M 5/31511; A61C 19/06

(57) ABSTRACT

An injector for bone regeneration is provided, including: a syringe; a needle having an embedding portion for embedding the needle in the syringe, an injecting portion integrally connected to the embedding portion and protruding from the syringe, and a channel penetrating through the embedding portion and the injecting portion for receiving an inducible bone regeneration material; and a pushing element having a rod that is less in diameter than the channel of the needle, slidably and axially disposed in the channel of the needle, for pushing the inducible bone regeneration material to move through the injecting portion and be injected into a bone portion of a patient, when the rod in the channel of the needle is pushed toward the injecting portion. The injector has a reduced size, and is beneficial for treatment of bone defects.

### BACKGROUND OF THE INVENTION

1. Field of the Invention

The present invention relates to injectors, and, more particularly, to an injector for bone regeneration.

2. Description of Related Art

Periodontal diseases such as periodontitis often cause alveolar bone defects, thus likely resulting in displacement or malposition of nearby teeth and even adversely affecting other healthy teeth. Clinically, guided bone regeneration (GBR) technology is the most common technology for repairing alveolar bones. However, the GBR technology has some drawbacks.

First, the GBR technology uses an implant such as a barrier membrane to prevent unwanted cells from entering an alveolar bone defect. However, the barrier membrane is excessively exposed to the periodontal tissue and not easy to care after surgery. To avoid infection caused by exposure of the barrier membrane, the patient needs to return multiple times to the hospital.

Second, to place the barrier membrane into the alveolar bone defect, a new wound will be created to the periodontal tissue, thus causing discomfort of the patient.

Third, after the alveolar bone defect is repaired through bone regeneration, a second surgery needs to be performed to remove the barrier membrane. However, the second surgery may damage the periodontal tissue and cause other complications.

FIG. 1A is a schematic planar exploded view of a conventional injector 1, and FIG. 1B is a schematic planar assembly view of the injector 1 of FIG. 1A.

Referring to FIGS. 1A and 1B, the injector 1 has a syringe 11, a needle 12 and a pushing element 13. The syringe 11 has a syringe body 111 having a receiving chamber 114 for receiving an injection solution 14, a connecting portion 112 for connecting the needle 12 to the syringe 11, an extending portion 113 extending from the syringe body 111, and an inlet end 115. The needle 12 has a needle base 121 connected to the connecting portion 112 of the syringe 11, and a needle body 122 having a channel 123. The pushing element 13 has a rod 131, an operating portion 132 and a protruding portion 133.

The length L1 of the syringe 11 is about 8.9 cm. The diameter Φ11 of the syringe body 111 of the syringe 11 is about 1.9 cm. The diameter Φ12 of the extending portion 113 is about 3.0 cm. The length L2 of the needle 12 is about 5.6 cm. The diameter Φ2 of the needle body 122 is about 0.1 cm. The length L3 of the pushing element 13 is about 9.3 cm. The diameter Φ3 of the operating portion 132 and the protruding portion 133 is about 1.8 cm. The length L of the injector 1 is about 14.9 cm. The height H and width of the injector 1 are about 3.0 cm.

When the injector 1 is used by a user, for example, a doctor, the injection solution 14 is placed in the receiving chamber 114 of the syringe body 111 and the pushing element 13 is inserted into the receiving chamber 114 of the syringe body 111 and pushed in a direction D1. As such, the injection solution 14 in the receiving chamber 114 is moved through the channel 123 of the needle body 122 and injected into the body of a patient.

However, the injector 1 is a common injector having a large size and not specially used for bone regeneration. The receiving chamber 114 of the syringe 11 has a large volume and is not suitable for a small amount of inducible bone regeneration material.

Therefore, there is a need to provide an injector for bone regeneration so as to overcome the above-described drawbacks.

### SUMMARY OF THE INVENTION

In view of the above-described drawbacks, the present invention provides an injector for bone regeneration. The injector has a minimized size, and is beneficial for treatment of bone defects.

The injector according to the present invention comprises: a syringe; a needle having an embedding portion for embedding the needle in the syringe, an injecting portion integrally connected to the embedding portion and protruding from the syringe, and a channel penetrating through the embedding portion for receiving an inducible bone regeneration material; and a pushing element having a rod that is less in diameter than the channel of the needle, slidably and axially disposed in the channel of the needle, for pushing the inducible bone regeneration material to move through the injecting portion and be injected into a bone portion of a patient, when the rod in the channel of the needle is pushed toward the injecting portion.

In an embodiment, the injector has a length ranging from 4.5 to 5.3 cm, a height ranging from 1.0 to 1.6 cm, and a width ranging from 1.0 to 1.6 cm.

In an embodiment, the injector has a guiding hole in communication with the channel of the needle for guiding the rod of the pushing element into the channel of the needle. In an embodiment, the guiding hole of the syringe is in the shape of a funnel with a wide top portion and a narrow bottom portion and thus has an inclined surface for guiding the rod of the pushing element into the channel of the needle.

In an embodiment, the injector has an inlet end and an outlet end, the guiding hole is positioned at the inlet end of the syringe, and the injecting portion of the needle is positioned at the outlet end of the syringe.

In an embodiment, the syringe has a syringe body and a first extending portion outwardly extending from the syringe and being adjacent or connected to the inlet end of the syringe. In an embodiment, the syringe further has a second extending portion outwardly extending from the syringe body of the syringe and separated from the first extending portion by a gap.

In an embodiment, the syringe has a through hole interconnecting the inlet end and the outlet end. The through hole is used for the embedding portion of the needle to be received or adhesively fixed therein.

In an embodiment, the needle and the rod of the pushing element is made of stainless steel.

In an embodiment, the pushing element further has an operating portion connected to the rod, and a third extending portion outwardly extending from the operating portion.

In an embodiment, the inducible bone regeneration material is pre-placed in the channel of the needle. In an embodiment, the inducible bone regeneration material is a bone graft material, a growth factor powder, an animal extract, a growth factor solution, or chitin.

According to the present invention, the embedding portion of the needle is embedded in the syringe, the inducible bone regeneration material is placed in the channel of the needle, and the rod of the pushing element is less in diameter than the channel of the needle.

Therefore, compared with the prior art, the present invention avoids exposure of an implant such as a barrier membrane and does not create a new wound to the bone portion, for example, the periodontal tissue, thereby alleviating discomfort of the patient and reducing the risk of infection. Also, the present invention dispenses with a second surgery and thus avoids another damage to the bone portion and generation of other complications.

Further, after the hard tissue defect of the bone portion is repaired, the regenerated hard tissue can be used as a base for such as a subsequent artificial dental implant and also used to prevent depression of the hard tissue and improve appearance and ease of maintenance.

Furthermore, the injector of the present invention is specially used for bone regeneration and has a minimized size that is only one half or one third of the conventional injector.

In addition, by placing the inducible bone regeneration material in the channel of the needle, the present invention avoids unnecessary waste and reduces the cost. Further, the inducible bone regeneration material can be pre-placed in the channel of the needle so as to save time for the user.

Also, the syringe of the present invention has a guiding hole and an inclined surface for quickly guiding the rod of the pushing element into the channel of the needle, thereby saving time for the user.

Furthermore, a portion of the rod can be pre-inserted into the channel of the needle so as to save the time for the user to insert the rod into the channel of the needle.

### BRIEF DESCRIPTION OF DRAWINGS

FIG. 1A is a schematic planar exploded view of a conventional injector;

FIG. 1B is a schematic planar assembly view of the conventional injector of FIG. 1A;

FIG. 2A is a schematic planar assembly view of a syringe and a needle of an injector according to the present invention;

FIG. 2B is a schematic planar side view of the syringe and the needle of the injector of FIG. 2A;

FIG. 2C is a schematic planar exploded view of the syringe and the needle of the injector of FIG. 2A;

FIG. 3A is a schematic planar side view of a pushing element of the injector according to the present invention;

FIG. 3B is another schematic planar side view of the pushing element of the injector of FIG. 3A;

FIGS. 4A to 4C are schematic planar views showing the use of the injector according to the present invention, wherein the injector combines the syringe and needle of FIG. 2A with the pushing element of FIG. 3A; and

FIGS. 5A to 5C are schematic planar views showing application of the injector according to the present invention to a bone portion of a patient.

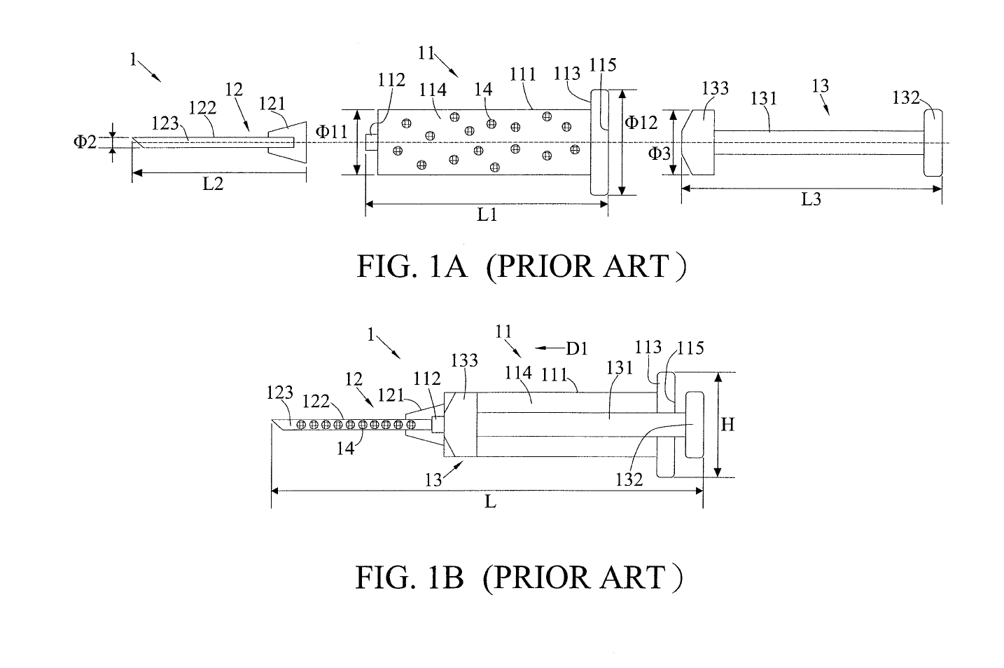

### DETAILED DESCRIPTION OF PREFERRED EMBODIMENTS

The following illustrative embodiments are provided to illustrate the disclosure of the present invention, these and other advantages and effects can be apparent to those in the art after reading this specification.

It should be noted that all the drawings are not intended to limit the present invention. Various modifications and variations can be made without departing from the spirit of the present invention. Further, terms such as “a”, “first”, “second”, “channel”, “embedded” etc. are merely for illustrative purposes and should not be construed to limit the scope of the present invention.

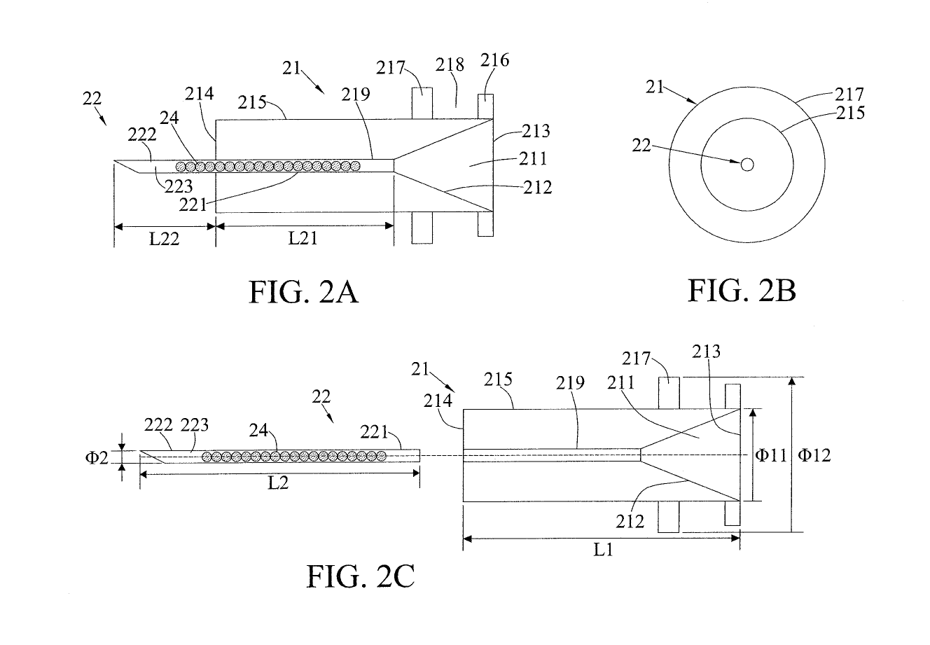

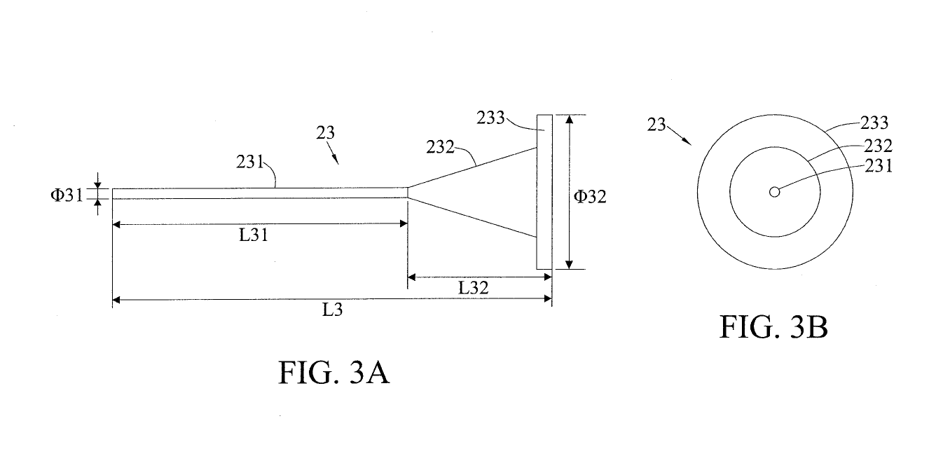

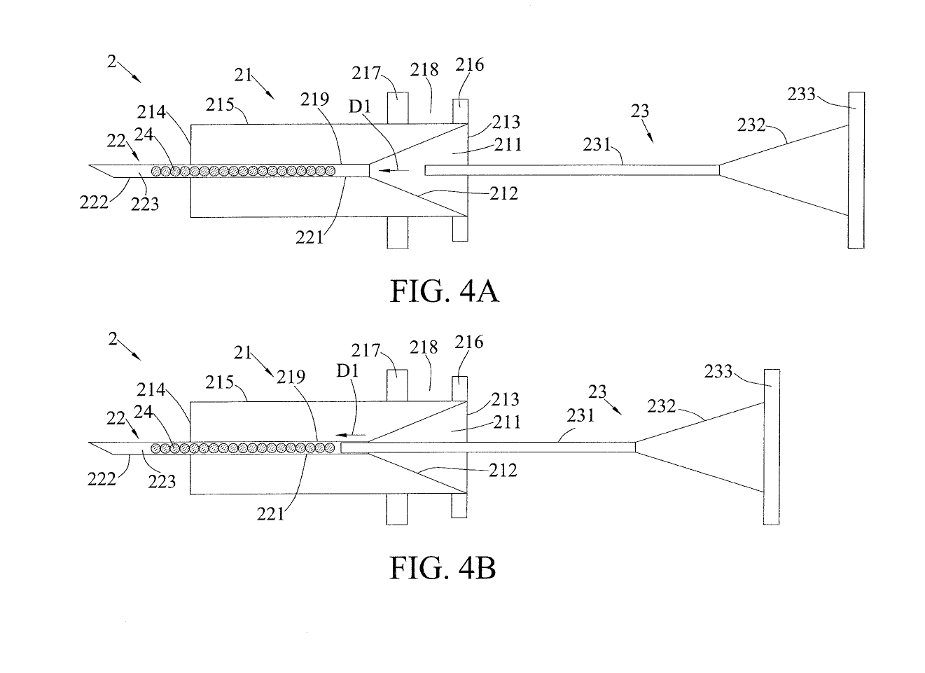

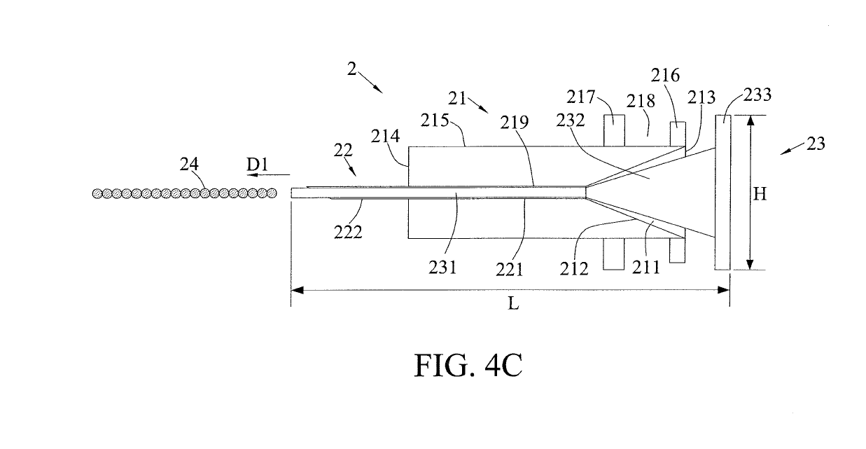

FIG. 2A is a schematic planar assembly view of a syringe 21 and a needle 22 of an injector 2 according to the present invention. FIG. 2B is a schematic planar side view of the syringe 21 and the needle 22 of the injector 2 of FIG. 2A, and FIG. 2C is a schematic planar exploded view of the syringe 21 and needle 22 of the injector 2 of FIG. 2A. FIG. 3A is a schematic planar side view of a pushing element 23 of the injector 2 according to the present invention, and FIG. 3B is another schematic planar side view of the pushing element 23 of the injector of FIG. 3A. FIGS. 4A to 4C are schematic planar views showing the use of the injector 2 according to the present invention, wherein the injector 2 combines the syringe 21 and the needle 22 of FIG. 2A with the pushing element 23 of FIG. 3A.

Referring to FIGS. 2A to 4C, the injector 2 has a syringe 21, a needle 22 and a pushing element 23.

Referring to FIGS. 2A to 2C, the needle 22 has an embedding portion 221 for embedding the needle 22 in the syringe 21, an injecting portion 222 integrally connected to the embedding portion 221, and a channel 223 penetrating through the embedding portion 221 and the injecting portion 222. The embedding portion 221 is embedded in the syringe 21 by engaging or adhering. The injecting portion 222 protrudes from the syringe 21. The channel 223 can receive an inducible bone regeneration material 24. The embedding portion 221 is, for example, a front portion of the needle 22, and the injecting portion 222 is, for example, a rear portion of the needle 22.

The inducible bone regeneration material 24 can be, but not limited to: (1) a solid material such as a bone graft material, for example, tricalcium phosphate (TCP), or a growth factor powder; (2) a liquid material such as an animal extract, for example, platelet rich plasma (PRP), or a growth factor solution; or (3) a colloid material such as chitin.

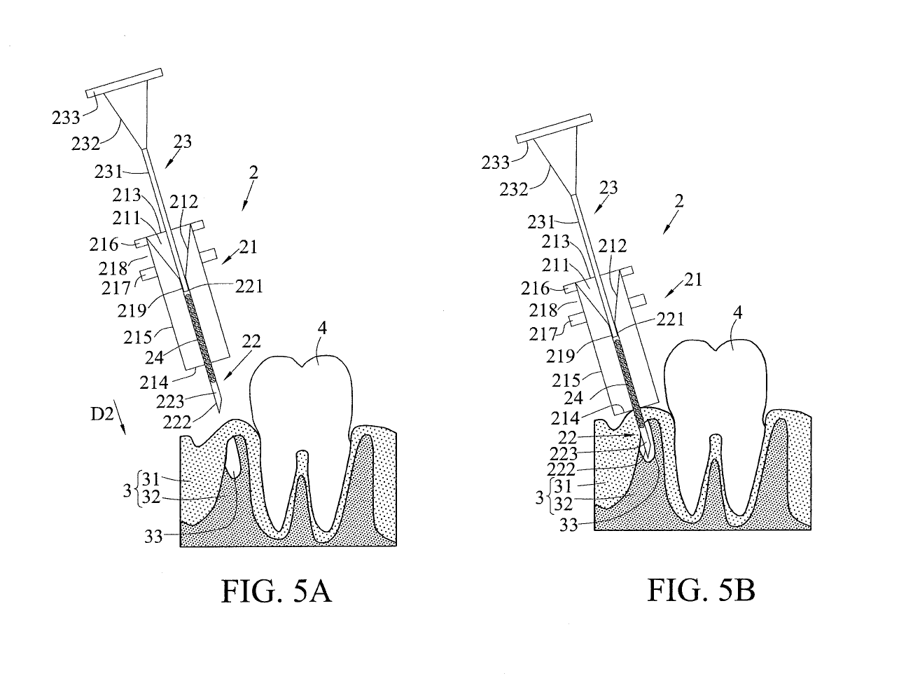

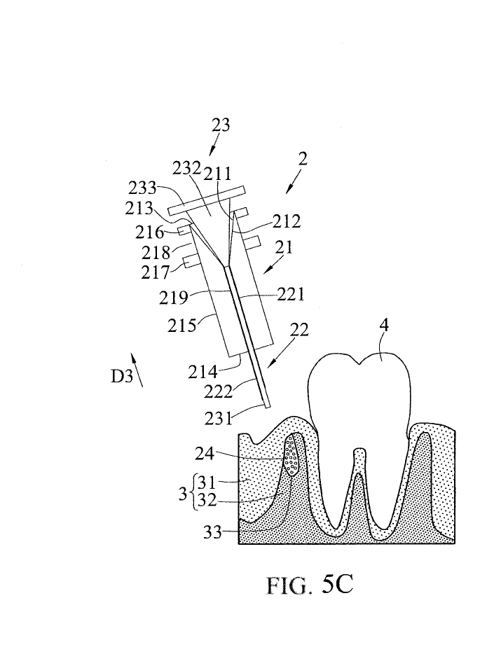

Referring to FIGS. 3A to 4C, the pushing element 23 can be a pusher. The pushing element 23 has a rod 231 slidably and axially disposed in the channel 223 of the needle 22. The diameter Φ31 of the rod 231 (as shown in FIG. 3A) is less than or slightly less than the diameter Φ2 of the channel 223 of the needle 22 (as shown in FIG. 2C). As such, when the rod 231 is pushed toward the injecting portion 222 along the channel 223, the inducible bone regeneration material 24 is moved through the injecting portion 222 and injected into a bone portion 3 of a patient (as shown in FIGS. 5A to 5C).

Referring to FIGS. 4A to 4C, the syringe 21 has a guiding hole 211 in communication with the channel 223 of the needle 22 for guiding the rod 231 of the pushing element 23 into the channel 223 in a direction D1. The guiding hole 211 of the syringe 21 is wide at top and narrow at bottom or in the shape of a tapered funnel. As such, the guiding hole 211 has an inclined surface 212, for the rod 231 of the pushing element 23 to be guided quickly into the channel 223 of the needle 22.

The syringe 21 further has an inlet end 213 and an outlet end 214. The guiding hole 211 is positioned at the inlet end 213 of the syringe 21, and the injecting portion 222 of the needle 22 is positioned at the outlet end 214 of the syringe 21. In an embodiment, the guiding hole 211 of the syringe 21 is recessed into the inlet end 213 of the syringe 21, and the injecting portion 222 of the needle 22 protrudes from the outlet end 214 of the syringe 21.

The syringe 21 has a syringe body 215 and a first extending portion 216 outwardly extending from the base 215. The first extending portion 216 is adjacent to or positioned on the inlet end 213 of the syringe 21 so as to be adjacent or connected to the guiding hole 211 of the syringe 21.

The syringe 21 further has a second extending portion 217 outwardly extending from the base 215. The second extending portion 217 is separated from the first extending portion 216 by a gap 218.

The syringe 21 further has a through hole 219 for the embedding portion 221 of the needle 22 to be received or adhesively fixed in the through hole 219.

Referring to FIGS. 3A and 3B, the pushing element 23 further has an operating portion 232 connected to the rod 231, and a third extending portion 233 outwardly extending from the operating portion 232. The operating portion 232 can have a tapered cone shape corresponding to the shape of the guiding hole 211 of the syringe 21.

In an embodiment, referring to FIGS. 2A to 2C, the length L1 of the syringe 21 is between 2.0 and 2.4 cm, for example, 2.2 cm, the width and height of the syringe 21 and the diameter Φ12 of the second extending portion 217 is between 1.0 and 1.6 cm, for example, 1.3 cm, the diameter Φ11 of the base 215 or the guiding hole 211 is between 0.3 and 0.7 cm, for example, 0.5 cm, the length L2 of the needle 22 is between 2.3 and 2.7 cm, for example, 2.5 cm, the diameter Φ2 of the needle 22 is between 0.105 and 0.108 cm, for example, 0.106 cm, the length L21 of the embedding portion 221 is between 1.3 and 1.5 cm, for example, 1.4 cm, and the length L22 of the injecting portion 222 is between 1.0 and 1.2 cm, for example, 1.1 cm.

Referring to FIGS. 3A to 3B, the length L3 of the pushing element 23 is between 4.5 and 5.3 cm, for example, 4.9 cm, the width and height of the pushing element 23 and the diameter Φ32 of the third extending portion 233 is between 1.0 and 1.6 cm, for example, 1.3 cm, the length L31 of the rod 231 is between 2.8 and 3.2 cm, for example, 3.0 cm, the diameter Φ31 of the rod 231 is between 0.08 and 0.09 cm, for example, 0.085 cm, the total length L32 of the operating portion 232 and the third extending portion 233 is between 1.7 and 2.1 cm, for example, 1.9 cm, the length of the operating portion 232 is between 1.6 and 1.8 cm, for example, 1.7 cm, and the length (i.e., thickness) of the first extending portion 216, the second extending portion 217 and the third extending portion 233 is between 0.1 and 0.3 cm, for example, 0.2 cm.

Referring to FIG. 4C, the length of the injector 2 is between 4.5 and 5.3 cm, for example, 4.9 cm, the height H and width of the injector 2 is between 1.0 and 1.6 cm, for example, 1.3 cm, and the length L31 of the rod 231 of FIG. 3A, which is between 2.8 and 3.2 cm, is slightly greater than or equal to the length L2 of the needle 22 of FIG. 2A, which is between 2.3 and 2.7 cm. As such, referring to FIG. 4C, driven by the rod 231, the inducible bone regeneration material 24 in the channel 223 of the needle 22 can be completely moved out of the needle 22.

In an embodiment, the needle 22 and the rod 231 of the pushing element 23 are made of stainless steel, and the syringe 21 and the operating portion 232 and the third extending portion 233 of the pushing element 23 are made of plastic such as polypropylene.

Referring to FIG. 4A, the inducible bone regeneration material 24 can be pre-placed in the channel 223 of the needle 22 so as to save the time for a user, for example, a doctor, to place the material 24 in the channel 22. In other embodiments, the inducible bone regeneration material 24 can be placed into the channel 223 of the needle 22 through the guiding hole 211 by a user who wants to use the needle 22.

Then, referring to FIGS. 4A and 4B, the user can place a plurality of fingers on the third extending portion 233 of the pushing element 23, on the gap 218 and the first extending portion 216 or the second extending portion 217 so as to insert the rod 231 of the pushing element 23 into the channel 223 of the needle 22 through the guiding hole 211 in the direction D1. In other embodiments, a portion of the rod 231 (for example, a front portion of the rod 231) can also be pre-inserted into the channel 223 of the needle 22 so as to save time for the user.

Thereafter, referring to FIG. 4C, the rod 231 of the pushing element 23 is partially or completely inserted into the channel 223 of the needle 22 so as to cause the operating portion 232 of the pushing element 23 to abut against the inclined surface 212 of the guiding hole 211 of the syringe 21. As such, the inducible bone regeneration material 24 in the channel 223 is partially or completely moved out of the injecting portion 222.

FIGS. 5A to 5C are schematic planar views showing application of the injector 2 to a bone portion 3 of a patient.

Referring to FIG. 5A, the injecting portion 222 is placed in a direction D2 to target a defect 33 of the bone portion 3.

In an embodiment, the bone portion 3 has soft tissue 31 and hard tissue 32 around a periphery of a tooth 4, for example, gum, periodontal ligament and alveolar bone. The inducible bone regeneration material 24 is placed in the defect 33 of the hard tissue 32 such as the alveolar bone of the bone portion 3. In other embodiments, the bone portion 3 can be a hand bone, a foot bone and so on.

Then, referring to FIG. 5B, the injecting portion 222 is inserted into the defect 33 of the bone portion 3 with the outlet end 214 of the syringe 21 abutting against the surface of the soft tissue 31.

Subsequently, referring to FIG. 5C, the rod 231 of the pushing element 23 is partially or completely inserted into the channel 223 of the needle 22 with the operating portion 232 of the pushing element 23 abutting against the inclined surface 212 of the guiding hole 211 of the syringe 21. As such, the inducible bone regeneration material 24 in the channel 223 is partially or completely injected into the defect 33 of the bone portion 3. Finally, the injector 2 as well as the needle 22 is withdrawn from the bone portion 3 in a direction D3. As such, the injecting process is completed.

According to the present invention, the embedding portion of the needle is embedded in the syringe, the inducible bone regeneration material is placed in the channel of the needle, and the rod of the pushing element is less in diameter than the channel of the needle.

Therefore, compared with the prior art, the present invention avoids exposure of an implant such as a barrier membrane and does not create a new wound to the bone portion, for example, the periodontal tissue, thereby alleviating discomfort of the patient and reducing the risk of infection. Also, the present invention dispenses with a second surgery and thus avoids another damage to the bone portion and generation of other complications.

Further, after the hard tissue defect of the bone portion is repaired, the regenerated hard tissue can be used as a base for such as a subsequent artificial dental implant and also used to prevent depression of the hard tissue and improve appearance and ease of maintenance.

Furthermore, the injector of the present invention is specially used for bone regeneration and has a minimized size that is only one half or one third of the conventional injector.

In addition, by placing the inducible bone regeneration material in the channel of the needle, the present invention avoids unnecessary waste and reduces the cost. Further, the inducible bone regeneration material can be pre-placed in the channel of the needle so as to save time for the user.

Also, the syringe of the present invention has a guiding hole and an inclined surface for quickly guiding the rod of the pushing element into the channel of the needle, thereby saving time for the user.

Furthermore, a portion of the rod can be pre-inserted into the channel of the needle so as to save the time for the user to insert the rod into the channel of the needle.

The above-described descriptions of the detailed embodiments are only to illustrate the preferred implementation according to the present invention, and it is not to limit the scope of the present invention. Accordingly, all modifications and variations completed by those with ordinary skill in the art should fall within the scope of present invention defined by the appended claims.

What is claimed is:

1. An injector for bone regeneration, comprising:
a syringe;
a needle having an embedding portion for embedding the needle in the syringe, an injecting portion integrally connected to the embedding portion and protruding from the syringe, and a channel penetrating through the embedding portion and the injecting portion for receiving an inducible bone regeneration material; and
a pushing element having a rod that is less in diameter than the channel of the needle, slidably and axially disposed in the channel of the needle, for pushing the inducible bone regeneration material to move through the injecting portion and be injected into a bone portion of a patient, when the rod in the channel of the needle is pushed toward the injecting portion.

2. The injector of claim 1, wherein the injector has a length of from 4.5 to 5.3 cm, a height of from 1.0 to 1.6 cm, and a width of from 1.0 to 1.6 cm.

3. The injector of claim 1, wherein the syringe has a guiding hole in communication with the channel of the needle for guiding the rod of the pushing element into the channel of the needle.

4. The injector of claim 3, wherein the guiding hole of the syringe is in a shape of a funnel with a wide top portion and a narrow bottom portion, and thus has an inclined surface for guiding the rod of the pushing element into the channel of the needle.

5. The injector of claim 3, wherein the syringe further has an inlet end and an outlet end, and the guiding hole of the syringe and the injecting portion of the needle are positioned at the inlet end and the outlet end of the syringe, respectively.

6. The injector of claim 3, wherein the syringe has a syringe body and a first extending portion outwardly extending from the syringe body and being adjacent or connected to the inlet end of the syringe.

7. The injector of claim 6, wherein the syringe further has a second extending portion outwardly extending from the syringe body and separated from the first extending portion by a gap.

8. The injector of claim 1, wherein the syringe is formed with a through hole interconnecting the inlet end and the outlet end of the syringe, for receiving or adhesively fixing the embedding portion of the needle in the syringe.

9. The injector of claim 1, wherein the needle and the rod of the pushing element are made of stainless steel.

10. The injector of claim 1, wherein the pushing element further has an operating portion connected to the rod, and a third extending portion outwardly extending from the operating portion.

11. The injector of claim 1, wherein the inducible bone regeneration material is pre-placed in the channel of the needle.

12. The injector of claim 1, wherein the inducible bone regeneration material is a bone graft material, a growth factor powder, an animal extract, a growth factor solution, or chitin.
